(19) United States
(12) Patent Application Publication
(10) Pub. No.: US 2007/0225548 A1
(43) Pub. Date: Sep. 27, 2007

(54) Direct flow sexual device

(76) Inventor: Diane T. Stelly, Houston, TX (US)

Correspondence Address: DELPHINE M. JAMES, 2656 SOUTH LOOP WEST #170, HOUSTON, TX, 77054, US

(21) Appl. No.: 11/359,998

(22) Filed: Feb. 22, 2006

(51) Int. Cl. A61F 5/00

(52) U.S. Cl. 600/38

(57) ABSTRACT

A sexual stimulation device comprises a tubular hose and a connector member. Tubular hose further comprises a substantially straight elongated cylindrical shape upper portion. The lower portion of tubular hose is slightly curved upward ending in a tip portion which is configured substantially similar to the tip of a male penis. At least one outlet is situated within the tip portion for directly water flow to stimulate the female clitoris. Connector member 20 is situated on top of the upper end of the tubular hose and is attached thereto. Connector member 20 is further adapted to attach to a faucet in at tub forming a water tight seal.

### BACKGROUND

The present invention relates to devices adapted to enhance sexual functioning and to provide sexual stimulation. More particularly this invention relates to devices that provide sexual stimulation using water. As women explore their sensuality, more women are discovering sexual devices which they can add to their masturbation practice. Vibrators are more popular sexual devices. They are inserted into the vagina to stimulate intercourse and stimulate the clitoris. In prior art there are several sexual devices. For example, US Patent No. 2004/0186344 which discloses a J shape vibrating device which is inserted into the vagina. However, the present invention is a unique sexual device which utilizes water to stimulate the clitoris.

### SUMMARY

It is an object of the present invention to provide an improved female sexual stimulation device.

It is further an object of the present invention to provide an improved sexual stimulation device that utilizes water for sexual stimulation.

In view of the foregoing objectives, the sexual stimulation device according to the present invention includes a hose configured as a penis that attaches to a water faucet in a bathtub. The tip of the sexual stimulation device further comprises small apertures for directing water flow into the clitoris. The invention includes other features and advantages which also enhance its ability to be quite useful and attractive. Such features and advantages will become apparent from the drawings in conjunction with the detailed description.

### BRIEF DESCRIPTION OF THE DRAWINGS

FIG. 1 is a perspective view of the sexual device according to the present invention installed in a bathroom; and

FIG. 2 is an enlarged perspective view of the sexual device according to the present invention.

### DETAILED DESCRIPTION

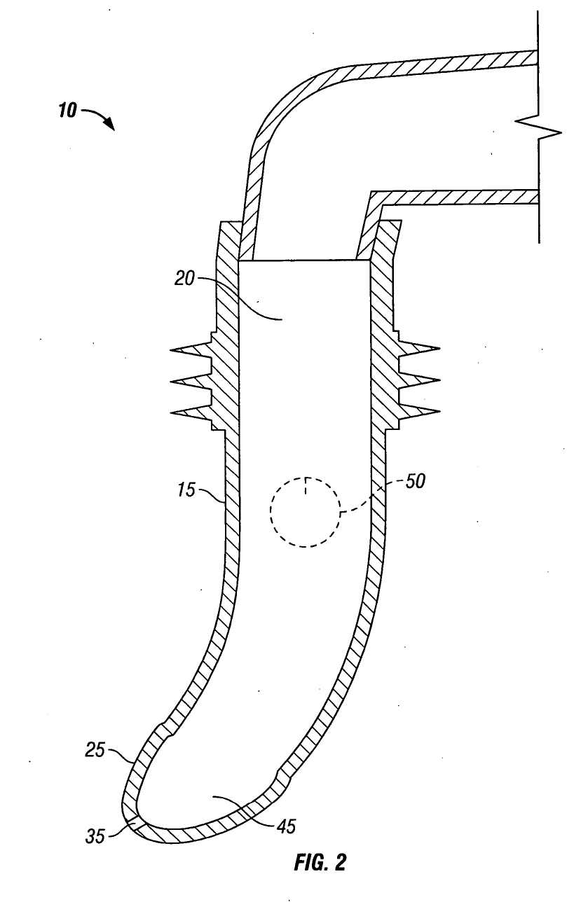

Referring to FIGS. 1-2, a sexual stimulation device according to the present invention is generally designated by numeral 10. The device 10 comprises a tubular hose 15 and a connector member 20.

Tubular hose 15 further includes a substantially straight elongated cylindrical shape upper portion. The lower portion of tubular hose 15 is slightly curved ending in tip portion 25. Tip portion 25 is configured substantially like the tip of a male penis. Tip portion 25 further includes at least one outlet 30 for directing water flow for clitoral stimulation. To allow water flow through tubular hose 15, the interior of tubular hose 15 is hollow. Additionally, tubular hose 15 is preferable made of a sturdy material such as plastic or another such suitable material and has an overall length of at least 10 inches.

Connector member 20 is situated on top of tubular hose 15 and attached thereto. As shown, connector member 20 has small cylindrical shape hollow portion adapted to attach the faucet of the tub. Preferably, connector member 20 is made of a flexible and stretchable material and has a length of preferably at least 2 inches. The durability of the material is such that a water tight flow is established upon attachment to the faucet. Additionally, a separator member 21 can be situated between connector member 20 and the upper end of hose 15. As shown separator member 21 is an aesthetic ornament added to the device.

As shown in FIG. 3, the present invention further includes at least one spray nozzle device 45 situated within tubular hose 15 directly into outlet 35. Control mechanism 50 is externally mounted onto the outer surface of tubular hose 15 and is operationally connected to spray nozzle device 45. Control mechanism 50 is adapted to control water flow through outlet 35 in a variety of ways i.e. direct stream, spray stream, drip stream, bursting stream, or another such suitable stream of water flow.

In use, as shown, connector member 20 attaches to faucet 40. The female lye in the tub with her leg spread apart such that as the water flow from faucet 40 the water is directed through outlet 35 directly into the clitoris for stimulation thereof.

What is claim is:

1. A sexual stimulation device comprising:
a hollow tubular hose having an upper portion and a lower portion; the upper portion having a substantially straight elongated cylindrical shape, the upper portion extending downward into a slight curved lower portion; the lower portion ending in a tip portion;
a connector member adapted to connect to a water faucet to form a water tight seal, the connector member securely attached to a top end of the upper portion of the tubular hose;
at least on outlet means strategically incorporated within the tip portion, the outlet means for directing the water flow from the water faucet;

2. The device of claim 1 wherein the tip portion is configured substantially similar to a tip of a male penis.

3. The device of claim 1 wherein the connector member has short hollow cylindrical shape and is made of a stretchable flexible material capable of forming a water tight seal.

4. The device of claim 1 further comprising a water flow mechanism internally situated with the lower portion of the hose over the outlet means, the water flow mechanism adapted to the pressure of the water flow from the faucet through the outlet means;
a control mechanism externally mounted at a strategic location on an outer surface area of the tubular hose, the control mechanism being operationally coupled to the water flow mechanism such a user can vary the pressure of the water flow through the outlet means to a desired stream.

5. The device of claim 4 wherein the desired stream is a dripping stream, bursting stream, spray stream, or a direct constant flow stream.

6. The device of claim 1 further comprises a separator member situated between the connector member and the top end of the tubular hose.
